(19) United States
(12) Patent Application Publication
(10) Pub. No.: US 2013/0190326 A1
(43) Pub. Date: Jul. 25, 2013

(54) Use of flibanserin for the treatment of post-menopausal sexual desire disorders

(71) Applicant: SPROUT PHARMACEUTICALS, INC., Raleigh, NC (US)

(72) Inventors: Stephane Pollentier, AW Schoorl (NL); Robert E. Pyke, New Fairfield, CT (US)

(73) Assignee: SPROUT PHARMACEUTICALS, INC., Raleigh, NC (US)

(21) Appl. No.: 13/674,486

(22) Filed: Nov. 12, 2012

Related U.S. Application Data
(63) Continuation of application No. 11/745,515, filed on May 8, 2007, now abandoned.
(60) Provisional application No. 60/830,987, filed on Jul. 14, 2006.
(60) Provisional application No. 60/746,817, filed on May 9, 2006.

(51) Int. Cl. A61K 31/496

(52) U.S. Cl. CPC A61K 31/496
USPC 514/254.06

(57) ABSTRACT

The invention relates to the use of flibanserin for the preparation of a medicament for the treatment of post-menopausal Sexual Desire Disorders.

### RELATED APPLICATIONS

This application claims the benefit of U.S. Provisional Application 60/746,817, filed May 9, 2006 and U.S. Provisional Application 60/830,987, filed Jul. 14, 2006, the disclosure of all of which are hereby incorporated by reference.

The invention relates to the methods for treatmenting post-menopausal Sexual Desire Disorders using flibanserin.

### DESCRIPTION OF THE INVENTION

The compound 1-[2-(4-(3-trifluoromethyl-phenyl)piperazin-1-yl)ethyl]-2,3-dihydro-1H-benzimidazol-2-one (flibanserin) is disclosed in form of its hydrochloride in European Patent Application EP-A-526434 and has the following chemical structure:

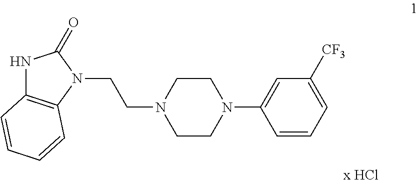 [SMILES: O=c1[nH]c2ccccc2n1CCN1CCN(c2cccc(C(F)(F)F)c2)CC1 ; "x HCl"]

Flibanserin shows affinity for the 5-HT_{1A} and 5-HT₂-receptor. It is therefore a promising therapeutic agent for the treatment of a variety of diseases, for instance depression, schizophrenia, and anxiety.

The generic term “Sexual Disorders” includes Sexual Desire Disorders, Sexual Arousal Disorders, Orgasmic Disorders, Sexual Pain Disorders, Sexual Dysfunction due to a General Medical Condition, Substance-Induced Sexual Dysfunction, and Sexual Dysfunction not otherwise specified (Diagnostic and Statistical Manual of Mental Disorders, 4th edition, Text Revision. Washington D.C., American Psychiatric Association, 2000).

The instant invention relates to the use of flibanserin, optionally in form of the free base, the pharmacologically acceptable acid addition salts and/or optionally in form of the hydrates and/or solvates thereof for the preparation of a medicament for the treatment of Sexual Desire Disorders in post-menopausal women.

Within the present invention the terms “treatment of post-menopausal Hypoactive Sexual Desire Disorder” etc. have the meaning of “treatment of Hypoactive Sexual Desire Disorders in post-menopausal women” etc.

The beneficial effects of flibanserin can be observed regardless of whether the Sexual Desire Disorder existed lifelong or was acquired, is of the “generalized type” or “situational type” and independent of etiologic origin (organic—both, physically and drug induced-, psychogen (due to psychological factors), a combination of organic—both, physically and drug induced-, and psychogen (due to psychological factors), or unknown). The term “lifelong” refers to such Sexual Desire Disorders of the present invention, which have been present since the onset of sexual functioning. The term “acquired” refers to such Sexual Desire Disorders of the present invention which developed only after a period of normal sexual functioning. The “generalized type” refers to such Sexual Disorders of the present invention wherein the disorder is not limited to certain types of stimulation, situations, or partners. The “situational type” applies to such Sexual Disorders of the present invention wherein the disorder is limited to certain types of stimulation, situations, or partners. The subtype due to “psychological factors” applies when psychological factors are judged to have the major role in the onset, severity, exacerbation, or maintenance of the Sexual Disorder, and general medical conditions and substance play no role in the etiology of the Sexual Disorder. Finally the subtype due to “combined factors” applies when 1) psychological factors are judged to have a role in the onset, severity, exacerbation, or maintenance of the Sexual Disorder, and 2) a general medical condition or substance use is also judged to be contributory but is not sufficient to account for a Sexual Disorder (Diagnostic and Statistical Manual of Mental Disorders, 4th edition, Text Revision. Washington D.C., American Psychiatric Association, 2000).

Therefore, e.g. the term “lifelong post-menopausal Hypoactive Sexual Desire Disorder” refers to Hypoactive Sexual Desire Disorder in post-menopausal women which has been present since the onset of sexual functioning and the term “acquired post-menopausal Hypoactive Sexual Desire Disorder” refers to Hypoactive Sexual Desire Disorder in post-menopausal women, which developed after a period of normal sexual functioning. Although there may seem to be an apparent contradiction in the wording “lifelong post-menopausal” this should be understood as a disorder diagnosed after the menopause whereby history reveals that the disorder in fact was present since the onset of sexual functioning.

Accordingly, in a preferred embodiment the invention relates to the use of flibanserin, optionally in form of the free base, the pharmacologically acceptable acid addition salts and/or optionally in form of the hydrates and/or solvates thereof for the preparation of a medicament for the treatment of disorders selected from the group consisting of lifelong post-menopausal Hypoactive Sexual Desire Disorder, lifelong post-menopausal Sexual Aversion Disorder, lifelong post-menopausal loss of sexual desire, lifelong post-menopausal lack of sexual desire, lifelong post-menopausal decreased sexual desire, lifelong post-menopausal inhibited sexual desire, lifelong post-menopausal loss of libido, lifelong post-menopausal libido disturbance, and lifelong post-menopausal frigidity.

Particular preferred according to the invention is the use of flibanserin, optionally in form of the free base, the pharmacologically acceptable acid addition salts and/or optionally in form of the hydrates and/or solvates thereof for the preparation of a medicament for the treatment of disorders selected from the group consiting of lifelong post-menopausal Hypoactive Sexual Desire Disorder, lifelong post-menopausal Sexual Aversion Disorder, lifelong post-menopausal loss of sexual desire, lifelong post-menopausal lack of sexual desire, lifelong post-menopausal decreased sexual desire, and lifelong post-menopausal inhibited sexual desire.

In a particularily preferred embodiment the invention relates to the use of flibanserin, optionally in form of the free base, the pharmacologically acceptable acid addition salts and/or optionally in form of the hydrates and/or solvates thereof for the preparation of a medicament for the treatment of disorders selected from the group of lifelong post-menopausal Hypoactive Sexual Desire Disorder lifelong post-menopausal loss of sexual desire and lifelong post-menopausal decreased sexual desire.

In a further preferred embodiment the invention relates to the use of flibanserin, optionally in form of the free base, the pharmacologically acceptable acid addition salts and/or optionally in form of the hydrates and/or solvates thereof for the preparation of a medicament for the treatment of disorders selected from the group consisting of acquired post-menopausal Hypoactive Sexual Desire Disorder, acquired post-menopausal Sexual Aversion Disorder, acquired post-menopausal loss of sexual desire, acquired post-menopausal lack of sexual desire, acquired post-menopausal decreased sexual desire, acquired post-menopausal inhibited sexual desire, acquired post-menopausal loss of libido, acquired post-menopausal libido disturbance, and acquired post-menopausal frigidity.

Furthermore preferred according to the invention is the use of flibanserin, optionally in form of the free base, the pharmacologically acceptable acid addition salts and/or optionally in form of the hydrates and/or solvates thereof for the preparation of a medicament for the treatment of disorders selected from the group consisting of acquired post-menopausal Hypoactive Sexual Desire Disorder, acquired post-menopausal Sexual Aversion Disorder, acquired post-menopausal loss of sexual desire, acquired post-menopausal lack of sexual desire, acquired post-menopausal decreased sexual desire, acquired post-menopausal inhibited sexual desire.

In a particularily preferred embodiment the invention relates to the use of flibanserin, optionally in form of the free base, the pharmacologically acceptable acid addition salts and/or optionally in form of the hydrates and/or solvates thereof for the preparation of a medicament for the treatment of disorders selected from the group of acquired post-menopausal Hypoactive Sexual Desire Disorder, acquired post-menopausal loss of sexual desire and acquired post-menopausal decreased sexual desire.

Furthermore the present invention relates to the generalized or situational subtype of any of the above mentioned conditions and/or to such which are due to psychological factors or due to combined factors.

Flibanserin can optionally used in form of the free base, in form of its pharmaceutically acceptable acid addition salts and/or optionally in form of the hydrates and/or solvates thereof. Suitable acid addition salts include for example those of the acids selected from, succinic acid, hydrobromic acid, acetic acid, fumaric acid, maleic acid, methanesulphonic acid, lactic acid, phosphoric acid, hydrochloric acid, sulphuric acid, tartaric acid and citric acid. Mixtures of the abovementioned acid addition salts may also be used. From the aforementioned acid addition salts the hydrochloride and the hydrobromide, particularily the hydrochloride, are preferred. If flibanserin is used in form of the free base, it is preferably used in form of flibanserin polymorph A as disclosed in WO 03/014079.

Flibanserin, optionally used in form of the free base, the pharmacologically acceptable acid addition salts and/or optionally in form of the hydrates and/or solvates thereof, may be incorporated into the conventional pharmaceutical preparation in solid, liquid or spray form. The composition may, for example, be presented in a form suitable for oral, rectal, parenteral administration or for nasal inhalation: preferred forms includes for example, capsules, tablets, coated tablets, ampoules, suppositories and nasal spray.

The active ingredient may be incorporated in excipients or carriers conventionally used in pharmaceutical compositions such as, for example, talc, arabic gum, lactose, gelatine, magnesium stearate, corn starch, acqueous or non acqueous vehicles, polyvynil pyrrolidone, semisynthetic glicerides of fatty acids, benzalconium chloride, sodium phosphate, EDTA, polysorbate 80. The compositions are advantageously formulated in dosage units, each dosage unit being adapted to supply a single dose of the active ingredient. The dosis range applicable per day is between 0.1 to 400, preferably between 1.0 to 300, more preferably between 2 to 200 mg. Each dosage unit may conveniently contain from 0.1 mg to 100 mg, preferably from 0.1 to 50 mg.

The dosage forms are administered to the patient 1, 2, 3, or 4 times daily. It is preferred that the compounds of the invention be administered either three or fewer times, more preferably once or twice daily consecutively over a period of time.

Preferably, the dose is administered to a patient in the morning and the evening, more preferably once in the morning (25 or 50 mg of flibanserin) and once in the evening (25 or 50 mg of flibanserin), most preferably once in the evening only (50 or 100 mg of flibanserin) consecutively over a period of time. In order to improve tolerability for a short period half the target dose can be administered.

As a result side-effects such as sedation are of lesser significance.

Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number or layers to achieve delayed release, possibly using the excipients mentioned above for the tablets.

Syrups or elixirs containing the active substances or combinations thereof according to the invention may additionally contain a sweetener such as saccharine, cyclamate, glycerol or sugar and a flavour enhancer, e.g of. a flavouring such as vanilline or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions for injection are prepared in the usual way, e.g of. with the addition of preservatives such as p-hydroxybenzoates, or stabilisers such as alkali metal salts of ethylenediamine tetraacetic acid, and transferred into injection vials or ampoules.

Capsules containing one or more active substances or combinations of active substances may for example be prepared by mixing the active substances with inert carriers such as lactose or sorbitol and packing them into gelatine capsules.

Suitable suppositories may be made for example by mixing with carriers provided for this purpose, such as neutral fats or polyethyleneglycol or the derivatives thereof.

The Examples which follow illustrate the present invention without restricting its scope:

### Examples of Pharmaceutical Formulations

| A) | Tablets | per tablet | |
|---|---|---|---|
| | flibanserin | 100 | mg |
| | lactose | 240 | mg |
| | corn starch | 340 | mg |
| | polyvinylpyrrolidone | 45 | mg |
| | magnesium stearate | 15 | mg |
| | | 740 | mg |

The finely ground active substance, lactose and some of the corn starch are mixed together. The mixture is screened, then moistened with a solution of polyvinylpyrrolidone in water, kneaded, wet-granulated and dried. The granules, the remaining corn starch and the magnesium stearate are screened and mixed together. The mixture is compressed to produce tablets of suitable shape and size.

| B) | Tablets | per tablet | |
|---|---|---|---|
| | flibanserin | 80 | mg |
| | corn starch | 190 | mg |
| | lactose | 55 | mg |
| | microcrystalline cellulose | 35 | mg |
| | polyvinylpyrrolidone | 15 | mg |
| | sodium-carboxymethyl starch | 23 | mg |
| | magnesium stearate | 2 | mg |
| | | 400 | mg |

The finely ground active substance, some of the corn starch, lactose, microcrystalline cellulose and polyvinylpyrrolidone are mixed together, the mixture is screened and worked with the remaining corn starch and water to form a granulate which is dried and screened. The sodium-carboxymethyl starch and the magnesium stearate are added and mixed in and the mixture is compressed to form tablets of a suitable size.

| C) | Coated tablets | per coated tablet | |
|---|---|---|---|
| | flibanserin | 5 | mg |
| | corn starch | 41.5 | mg |
| | lactose | 30 | mg |
| | polyvinylpyrrolidone | 3 | mg |
| | magnesium stearate | 0.5 | mg |
| | | 80 | mg |

The active substance, corn starch, lactose and polyvinylpyrrolidone are thoroughly mixed and moistened with water. The moist mass is pushed through a screen with a 1 mm mesh size, dried at about 45° C. and the granules are then passed through the same screen. After the magnesium stearate has been mixed in, convex tablet cores with a diameter of 6 mm are compressed in a tablet-making machine. The tablet cores thus produced are coated in known manner with a covering consisting essentially of sugar and talc. The finished coated tablets are polished with wax.

| D) | Capsules | per capsule | |
|---|---|---|---|
| | flibanserin | 150 | mg |
| | Corn starch | 268.5 | mg |
| | Magnesium stearate | 1.5 | mg |
| | | 420 | mg |

The substance and corn starch are mixed and moistened with water. The moist mass is screened and dried. The dry granules are screened and mixed with magnesium stearate. The finished mixture is packed into size 1 hard gelatine capsules.

| E) | Ampoule solution | | |
|---|---|---|---|
| | flibanserin | 50 | mg |
| | sodium chloride | 50 | mg |
| | water for inj. | 5 | ml |

The active substance is dissolved in water at its own pH or optionally at pH 5.5 to 6.5 and sodium chloride is added to make it isotonic. The solution obtained is filtered free from pyrogens and the filtrate is transferred under aseptic conditions into ampoules which are then sterilised and sealed by fusion.

| F) | Suppositories | | |
|---|---|---|---|
| | flibanserin | 50 | mg |
| | solid fat | 1650 | mg |
| | | 1700 | mg |

The hard fat is melted. At 40° C. the ground active substance is homogeneously dispersed. It is cooled to 38° C. and poured into slightly chilled suppository moulds.

1. A method of treating post-menopausal sexual desire disorders (lifelong or acquired) in a woman, comprising administering an effective amount of flibanserin, once daily, optionally in the form of the free base or a pharmacologically acceptable acid addition salt, to a woman in need thereof.

2. (canceled)

3. The method according to claim 1, wherein the post-menopausal sexual desire disorder is selected from the group consisting of lifelong post-menopausal Hypoactive Sexual Desire Disorder, lifelong post-menopausal Sexual Aversion Disorder, lifelong post-menopausal loss of sexual desire, lifelong post-menopausal lack of sexual desire, lifelong post-menopausal decreased sexual desire, lifelong post-menopausal inhibited sexual desire, lifelong post-menopausal loss of libido, lifelong post-menopausal libido disturbance, and lifelong post-menopausal frigidity.

4. The method according to claim 1, wherein the post-menopausal sexual desire disorder is selected from the group consisting of lifelong post-menopausal Hypoactive Sexual Desire Disorder, lifelong post-menopausal Sexual Aversion Disorder, lifelong post-menopausal loss of sexual desire, lifelong post-menopausal lack of sexual desire, lifelong post-menopausal decreased sexual desire, and lifelong post-menopausal inhibited sexual desire.

5. The method according to claim 1, wherein the post-menopausal sexual desire disorder is selected from the group consisting of acquired post-menopausal Hypoactive Sexual Desire Disorder, acquired post-menopausal Sexual Aversion Disorder, acquired post-menopausal loss of sexual desire, acquired post-menopausal lack of sexual desire, acquired post-menopausal decreased sexual desire, acquired post-menopausal inhibited sexual desire, acquired post-menopausal loss of libido, acquired post-menopausal libido disturbance, and acquired post-menopausal frigidity.

6. The method according to claim 1, wherein the post-menopausal sexual desire disorder is selected from the group consisting of acquired post-menopausal Hypoactive Sexual Desire Disorder, acquired post-menopausal Sexual Aversion Disorder, acquired post-menopausal loss of sexual desire, acquired post-menopausal lack of sexual desire, acquired post-menopausal decreased sexual desire, acquired post-menopausal inhibited sexual desire.

7. The method according claim 1, wherein the post-menopausal sexual desire disorders is of the generalized subtype.

8. The method according to claim 1, wherein the post-menopausal sexual desire disorders is of the situational subtype.

9. The method according to claim 1, wherein the post-menopausal sexual desire disorders is due to psychological factors.

10. The method according to claim 1, wherein the post-menopausal sexual desire disorders is due to combined factors.

11. The method according to claim 1, wherein flibanserin is administered in the form of a pharmaceutically acceptable acid addition salt wherein the salt is formed from an acid selected from the group consisting of succinic acid, hydrobromic acid, acetic acid, fumaric acid, maleic acid, methanesulphonic acid, lactic acid, phosphoric acid, hydrochloric acid, sulphuric acid, tartaric acid, citric acid, and mixtures thereof.

12. The method according to claim 1, wherein flibanserin is administered in the form of the free base.

13. The method according to claim 12, wherein flibanserin is administered in the form of a polymorph A of the free base, having a melting point of about 161° C. as measured using DSC.

14. The method according to claim 1, characterized in that flibanserin is administered in a dosage range between 0.1 to 400 mg per day.

15-18. (canceled)

19. The method of claim 14, wherein flibanserin is administered in a dosage range between 50 to 100 mg.

20. The method of claim 1, wherein flibanserin is administered once daily in the evening.

21. The method of claim 19, wherein said once daily administered flibanserin is in form of a polymorph A of the free base, having a melting point of about 161 oc as measured using DSC.

22. The method of claim 21, wherein said once daily administration is in the evening.

23. The method of claim 19 wherein the once daily dosage is about 100 mg.
